(19) United States
(12) Patent Application Publication
(10) Pub. No.: US 2017/0281910 A1
(43) Pub. Date: Oct. 5, 2017

(54) GUIDEWIRE DEVICE WITH DEPLOYABLE DISTAL END PORTION, SYSTEMS AND METHODS THEREOF

(71) Applicant: SafeRail Incorporated, Salt Lake City, UT (US)

(72) Inventors: Brian K. Whisenant, Salt Lake City, UT (US); Clark C. Davis, Holladay, UT (US)

(21) Appl. No.: 15/465,575

(22) Filed: Mar. 21, 2017

Related U.S. Application Data
(60) Provisional application No. 62/316,572, filed on Apr. 1, 2016.

(51) Int. Cl. A61M 25/09

(52) U.S. Cl. CPC A61M 25/0905; A61M 2210/125; A61M 2025/09175

(57) ABSTRACT

Devices, methods and systems are provided for delivering a guidewire to a target location within an anatomic structure. In one embodiment, a guidewire includes a guidewire body and a deployable guidewire end portion. The deployable guidewire end portion is coupled to a distal end of the guidewire body, the deployable guidewire end portion having a self-expandable structure moveable between a constricted position and an expanded position. With this arrangement, the guidewire end portion, in the expanded position, includes a distal most side surface sized and configured to a traumatically position and brace the guidewire end portion against tissue at the target location.

### CROSS-REFERENCE TO RELATED APPLICATIONS

The present application claims the benefit of U.S. Provisional Patent Application No. 62/316,572, filed on Apr. 1, 2016, the disclosure of which is hereby incorporated by reference in its entirety.

### TECHNICAL FIELD

The present invention relates generally to guidewires. More specifically, the present invention relates to guidewires for navigating vascular and cardiac structures.

### BACKGROUND

There are a number of medical conditions that necessitate physician intervention by catheter in order to provide diagnosis of or therapy for diseases of the vascular or cardiac systems. In many of the patients that require this type of treatment, the nature of their disease is such that their anatomy does not allow for easy or safe passage of catheters through the vasculature or to the target location. In these cases, a guidewire is often used to reach the target location followed by the catheter that tracks over that guidewire.

An additional risk to the interventional procedure involves the interaction of that guidewire with the anatomy of the patient. During the advancement or retraction of the catheter over the wire, or during the treatment procedure itself, the wire is often in contact with fragile anatomic structures such as arteries, veins or the valves or chambers of the heart.

In the case of transcatheter valve replacement (TVR), there is a need to place a relatively stiff guidewire across the native valve, such as the aortic valve, mitral valve or tricuspid valve, in order to guide the placement of the valve delivery system. The interaction of this relatively stiff guidewire with a delivery system that can also be quite stiff results in the potential for significant application of force at the distal end of the delivery system and in particular on the distal end of the guidewire.

Guidewires commonly used for these types of procedures are necessarily quite small in diameter, as they are intended to be passed through a small diameter lumen in the delivery system or through a sheath with a small inner diameter. The guidewire sizes typically used in an interventional cardiology procedure range from 0.014″ to 0.038″ in diameter. For a TVR implant procedure, a 0.035″ diameter guidewire is most commonly used. Due to the small size of these wires and therefore the limited surface contact that they have with the tissue at the distal end of the system, there is great potential for a high load to be transferred to fragile tissue at focal points during the use of a guidewire-based system.

To counteract some of the concerns related to the transfer of force to vulnerable tissue, guidewires are typically designed to taper in stiffness from the proximal end to the distal end, in order to reduce the risk of damage to the vasculature. There are a great number of tip configurations that have been developed to provide a combination of functional stiffness, steerability and distal softness. Generally, this is accomplished by designing a guidewire with a tapered core wire that is contained within an outer coil affixed to each end of the core wire. This provides some body and kink resistance to the wire while maintaining flexibility. While this type of design allows the guidewire to flex and distort in an attempt to manage distal displacement of the guidewire, it does not control force application.

The safety benefit of using a guidewire during an interventional cardiology procedure is well-established, both as an aid in navigating difficult anatomy and in stabilizing a catheter. However, a guidewire design has yet to be developed that substantially reduces the risk caused by translating force application to tissues during guidewire use.

### BRIEF SUMMARY OF THE INVENTION

Embodiments of the present invention are directed to various devices, systems and methods of positioning and bracing a guidewire at a target location in an anatomic structure. For example, in accordance with one embodiment of the present invention, the guidewire includes a guidewire body and a deployable guidewire end portion. The deployable guidewire end portion is coupled to a distal end of the guidewire body. With this arrangement, the deployable guidewire end portion includes a self-expandable structure that is moveable between a constricted position and an expanded position.

In one embodiment, the guidewire end portion includes multiple wires, the multiple wires being woven together. In another embodiment, upon the guidewire end portion being moved to the expanded position, the guidewire end portion extends to proximate at least one pad structure. In still another embodiment, upon the guidewire end portion being moved to the expanded position, the guidewire end portion extends to proximate multiple in-line pads. In another embodiment, the guidewire end portion includes multiple wires extending to proximate at least one pad structure.

In a further embodiment, the multiple in-line pads include a first pad and a second pad, the first pad being more distal than the second pad, the first pad having a larger diameter than the second pad. In still a further embodiment, the second pad is configured to be pressed against the first pad, the first pad having a distal side surface configured to be positioned against tissue. In yet a further embodiment, the multiple in-line pads include a first pad, a second pad, and a third pad. In another further embodiment, the first pad includes a larger diameter than the second pad and the third pad, the first pad being more distal than the first and second pads. In another embodiment, the multiple in-line pads include multiple wires, the multiple wires being woven together and extending to proximate the multiple in-line pads.

In another embodiment, the guidewire end portion includes a super-elastic material. In another embodiment, upon the guidewire end portion being moved to the expanded position, the guidewire end portion extends with a nesting pad structure. In still another embodiment, upon the guidewire end portion being moved to the expanded position, the guidewire end portion includes a distal side surface having a lateral width that is at least five times larger than a width of the guidewire body. In yet another embodiment, the guidewire end portion is actuatable between the constricted position and the expanded position.

In accordance with another embodiment of the present invention, a guidewire system includes a catheter and a guidewire. The guidewire includes a guidewire body and a guidewire end portion. The guidewire body extends between a proximal end and a distal end thereof. The guidewire end portion is coupled to the distal end of the guidewire body. The guidewire end portion includes a self-expandable structure that is moveable between a constricted position and an expanded position such that the guidewire end portion moves to the constricted position upon being positioned within a lumen of the catheter and the guidewire end portion is configured to self-expand to the expanded position upon being moved from within a catheter distal end of the catheter.

In one embodiment, the guidewire end portion includes multiple wires, the multiple wires being woven together and extending to proximate a pad structure. In another embodiment, upon the guidewire end portion moving to the expanded position, the guidewire end portion extends to proximate at least one pad structure. In still another embodiment, upon the guidewire end portion moving to the expanded position, the guidewire end portion extends to proximate multiple in-line pad structures. In yet another embodiment, the guidewire end portion includes a super-elastic material.

In another embodiment, upon the guidewire end portion moving to the expanded position, the guidewire end portion extends to proximate a nesting pad structure. In another embodiment, upon the guidewire end portion being moved to the expanded position, the guidewire end portion includes a distal side surface having a lateral width that is at least five times larger than a width of the guidewire body. In still another embodiment, the guidewire end portion is actuatable between the constricted position and the expanded position.

In accordance with another embodiment of the present invention, a method of positioning a guidewire at a target location is provided. The method includes the steps of: positioning a distal end of a catheter adjacent the target location; advancing a guidewire through a lumen of the catheter, the guidewire including a guidewire body and a guidewire end portion, the guidewire end portion coupled to a distal end of the guidewire body; and deploying the guidewire end portion from the distal end of the catheter such that the guidewire end portion includes a self-expandable structure to move from a constricted position within the catheter to a deployed, expanded position, the deployed, expanded position; wherein, upon deploying the guidewire end portion, bracing a distal most side surface of the guidewire end portion against tissue adjacent the target location.

In one embodiment, the method step of deploying includes deploying multiple wires of the guidewire end portion to extend and proximate a pad structure. In another embodiment, the method step of deploying includes deploying the guidewire end portion to expand and extend to proximate at least one pad structure. In still another embodiment, the method step of deploying includes deploying the guidewire end portion to expand and extend to proximate multiple in-line pad structures.

In another embodiment, the method step of deploying includes actuating the guidewire end portion from the constricted position to the expanded position. In another embodiment, the method step of deploying includes deploying the guidewire end portion to expand and extend to proximate a nesting pad structure. In still another embodiment, the method step of deploying includes deploying the guidewire end portion having the distal most side surface such that the distal most side surface includes a lateral width that is at least five times larger than a width of the guidewire body. In another embodiment, the method step of bracing includes placing a distal force on the guidewire to facilitate deploying an implant from a device catheter positioned over the guidewire.

### BRIEF DESCRIPTION OF THE SEVERAL VIEWS OF THE DRAWINGS

The foregoing and other advantages of the invention will become apparent upon reading the following detailed description and upon reference to the drawings in which:

FIG. 1 is a perspective view of a guidewire system, depicting a catheter and a guidewire such that a guidewire end portion is in a constricted position within the catheter, according to an embodiment of the present invention;

FIG. lA is an enlarged perspective view of a guidewire system, depicting the guidewire end portion in an expanded position, according to another embodiment of the present invention;

FIG. 2 is a simplified side view of the guidewire system, depicting the guidewire end portion in the expanded position in a simplified cross-sectional profile view, according to another embodiment of the present invention;

FIG. 3A is a simplified side view of another embodiment of a guidewire end portion of a guidewire system, depicting the guidewire end portion in the expanded position in a simplified cross-sectional profile view such that the guidewire end portion includes two pad portions, according to the present invention;

FIG. 3B is a simplified side view of another embodiment of a guidewire end portion of a guidewire system, depicting the guidewire end portion in the expanded position in a simplified cross-sectional profile view such that the guidewire end portion includes a single pad portion, according to the present invention;

FIG. 4A is a cross-sectional side view of a proximal coupling between the guidewire end portion and the guidewire, according to another embodiment of the present invention;

FIG. 4B is a cross-sectional side view of another embodiment of a proximal coupling between the guidewire end portion and the guidewire, according to the present invention;

FIG. 5 is a cross-sectional side view of a distal coupling of the guidewire end portion, according to another embodiment of the present invention;

FIG. 6 is a simplified side view of another embodiment of a guidewire end portion, depicting the guidewire end portion in the expanded position in a simplified cross-sectional profile view, the guidewire end portion not including a distal hub, according to the present invention;

FIG. 6A is a side view of one of the wires along the distal side of the guidewire end portion, depicting the one wire having a thinned portion, according to another embodiment of the present invention;

FIG. 7 is a simplified cross-sectional view of another embodiment of a guidewire system, depicting a guidewire end portion in a simplified cross-sectional profile view such that the guidewire end portion is actuatable, according to the present invention;

FIG. 8A is a simplified view of the guidewire system positioned in the heart, depicting the guidewire system positioned through a valve with the guidewire end portion in the constricted position, according to another embodiment of the invention;

FIG. 8B is a simplified view of the guidewire system positioned in the heart, depicting the guidewire end portion moved to the expanded position and positioned against tissue in the heart, according to another embodiment of the present invention;

FIG. 8C is a simplified view of the guidewire system positioned in the heart, depicting a catheter being withdrawn from the guidewire and heart, according to another embodiment of the present invention;

FIG. 8D is a simplified view of the guidewire with the deployed guidewire end portion positioned in the heart, depicting a device catheter positioned over the guidewire and the guidewire end portion being braced against tissue in the heart, according to another embodiment of the present invention;

FIG. 9 is a simplified view of the guidewire system and device catheter positioned through a mitral valve in the heart, depicting the deployed guidewire end portion positioned against tissue in the left ventricle of the heart, according to another embodiment of the present invention; and

FIG. 10 is a simplified view of the guidewire system and device catheter positioned through the tricuspid valve in the heart, depicting the deployed guidewire end portion positioned against tissue in the right ventricle of the heart, according to another embodiment of the present invention.

### DETAILED DESCRIPTION OF THE INVENTION

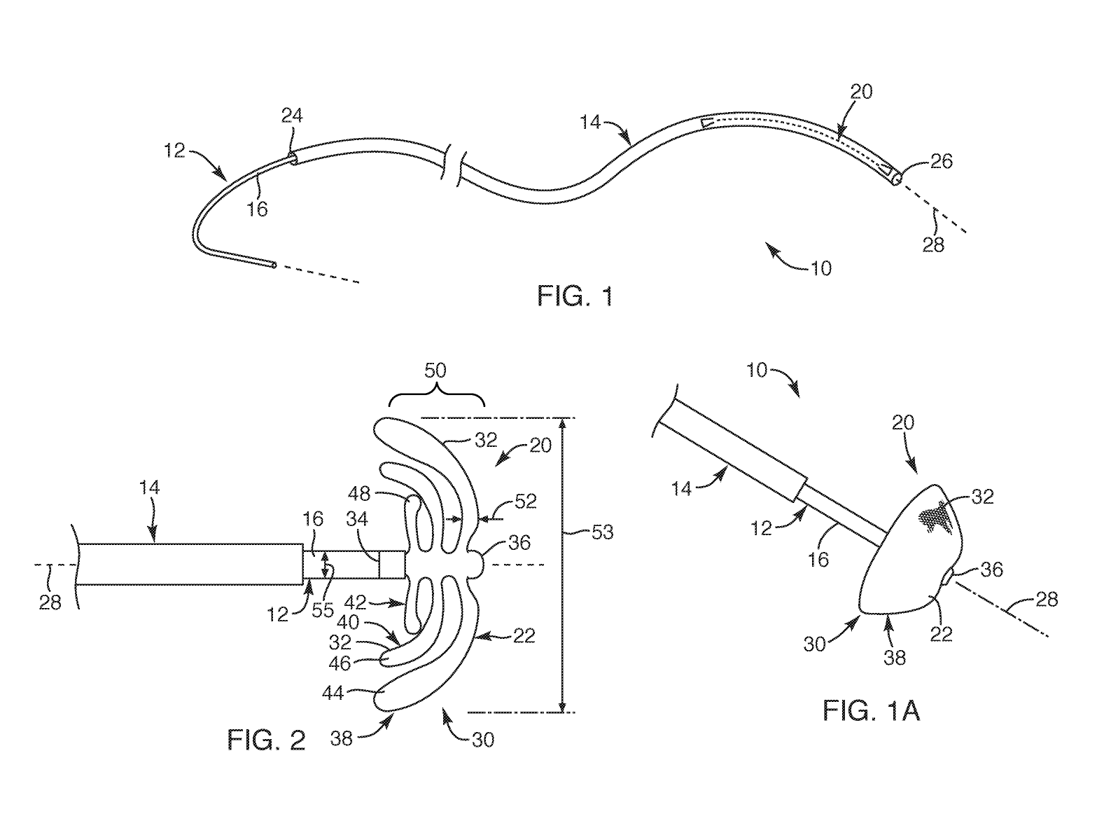

Referring to FIGS. 1 and 1A, simplified views of a guidewire system 10 with a guidewire end portion 20 in respective constricted and expanded positions are provided. In one embodiment, the guidewire system 10 may be employed to deploy and brace the guidewire end portion 20 against tissue in the heart in an a traumatic manner so as to substantially minimize any damage to tissue in the heart. The guidewire system 10 may include a guidewire 12 sized and configured to extend and move through a lumen of a catheter 14 as well as be employed for advancing other devices or catheters (not shown) over the guidewire 12.

The guidewire 12 may include a guidewire body 16 with the guidewire end portion 20 coupled to a distal end portion or distal end of the guidewire body 16. The guidewire end portion 20 may be a self-expandable structure. In another embodiment, the guidewire end portion 20 may be self-expandable and actuatable between the constricted and expanded positions. With this arrangement, the guidewire end portion 20 may be deployable so as to be moveable to the constricted position within the lumen of the catheter 14 and, upon the catheter 14 being moved proximally relative to the guidewire body 16, the guidewire end portion 20 may self-expand and move to the expanded position such that the guidewire end portion 20 exhibits a distal side surface 22 or distal side. The distal side surface 22 may be an enlarged area configured to brace against tissue, diffuse any force placed on the guidewire 12, and mechanically obstruct the guidewire body 16 from piercing or damaging the tissue.

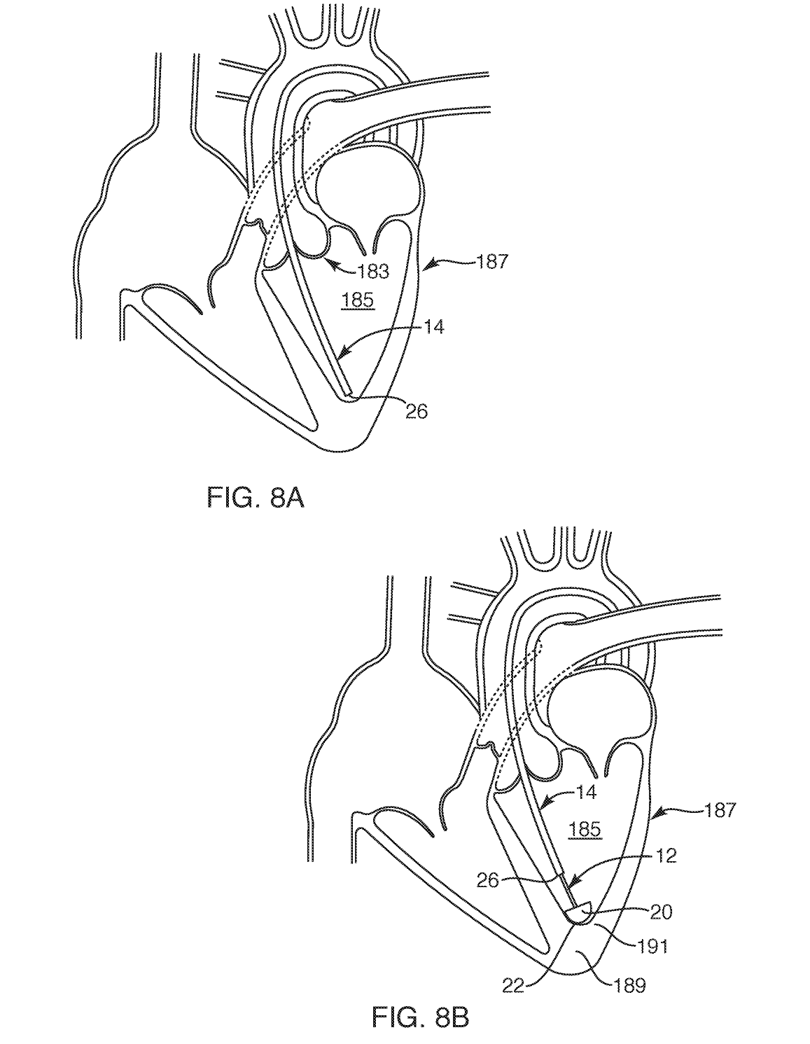

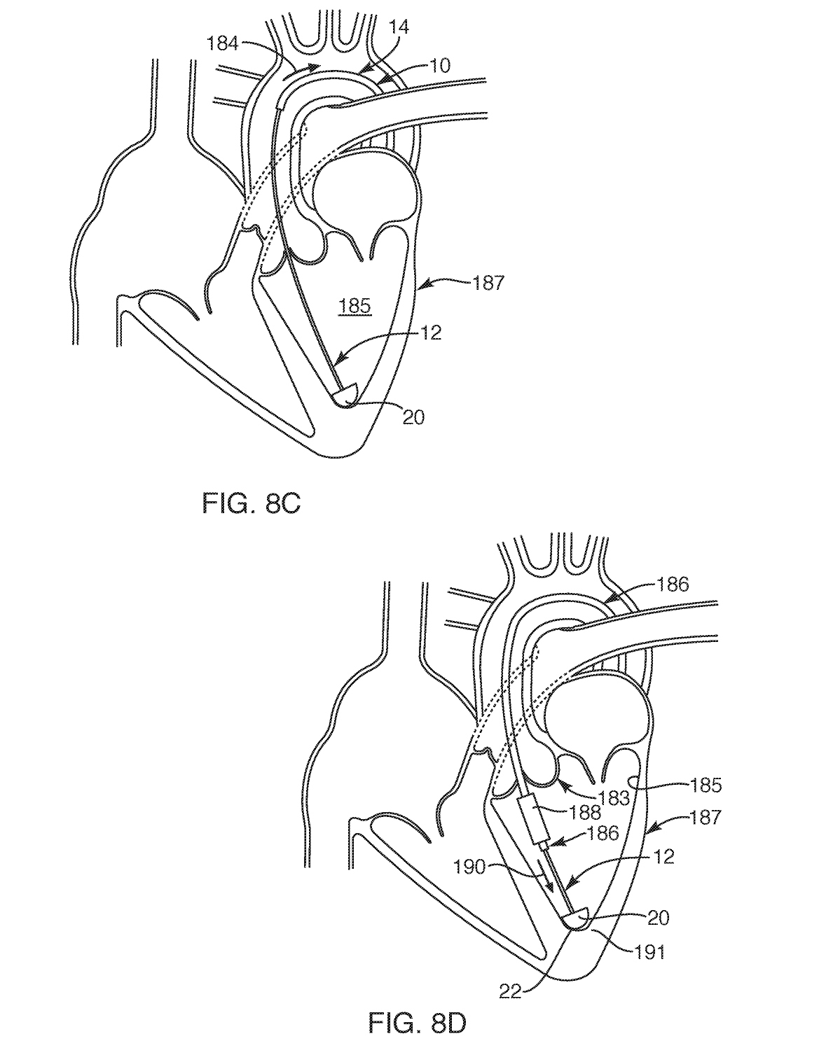

With the guidewire end portion 20 in the expanded position, the catheter 14 may be withdrawn from over the guidewire body 16. Once the catheter 14 is removed, a device catheter with an implant may be moved over the guidewire body 16 to position the implant within, for example, an aortic valve (see FIG. 8D) or any other suitable valve, such as a mitral valve or a tricuspid valve. To effectively position the implant in a valve, a physician may place a distal force on the guidewire 12. The distal side surface 22 of the guidewire end portion 20 being braced against the tissue acts to spread, distribute and diffuse the distal force across or along the distal side surface 22 being braced and forced against the tissue in the heart. In this manner, the guidewire 12 may be employed by a physician to place the necessary force against the tissue to position an implant with a device catheter over the guidewire 12 in a non-damaging, a traumatic manner to the tissue in the heart.

As set forth, the guidewire system 10 may include the catheter 14 or a sheath. Such catheter 14 may be a diagnostic catheter. The catheter 14 may be elongated with a catheter length that may be shorter than the guidewire. The catheter 14 may include a lumen extending through and between a proximal end 24 and a distal end 26 of the catheter 14. The lumen of the catheter 14 may be sized and configured to facilitate moving the guidewire 12 therethrough such that the guidewire end portion 20 may be positioned within and adjacent the distal end 26 of the catheter 14 in the constricted position.

Further, the guidewire 12 may define an axis 28 along a longitudinal length of the guidewire 12, the guidewire body 16 and the guidewire end portion 20 extending along the axis 28. The guidewire body 16 may include a longer length than the catheter 14. The guidewire body 16 may include guidewire components similar to typical guidewires, such as including a core wire disposed within a coil structure, and/or any other typical guidewire components or integrated guidewire structure. As set forth, the guidewire end portion 20 may be coupled to a distal end or distal end portion of the guidewire body 16. Such guidewire end portion 20 may include one or more pad portions or structures 30 having the distal side surface 22.

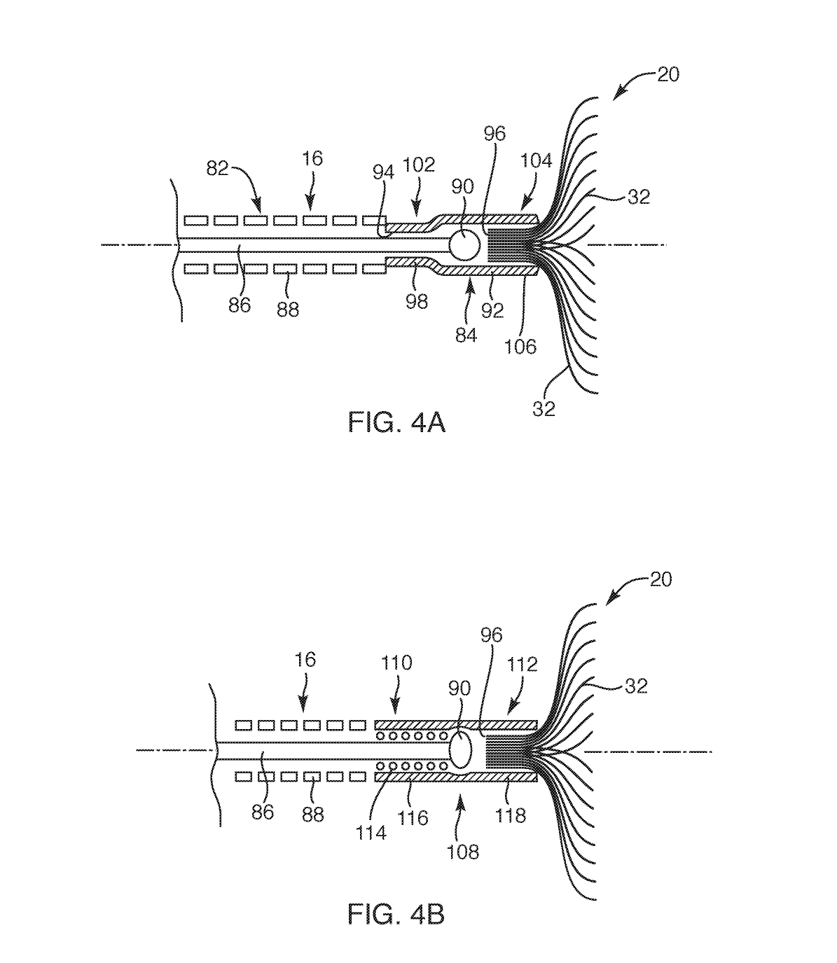

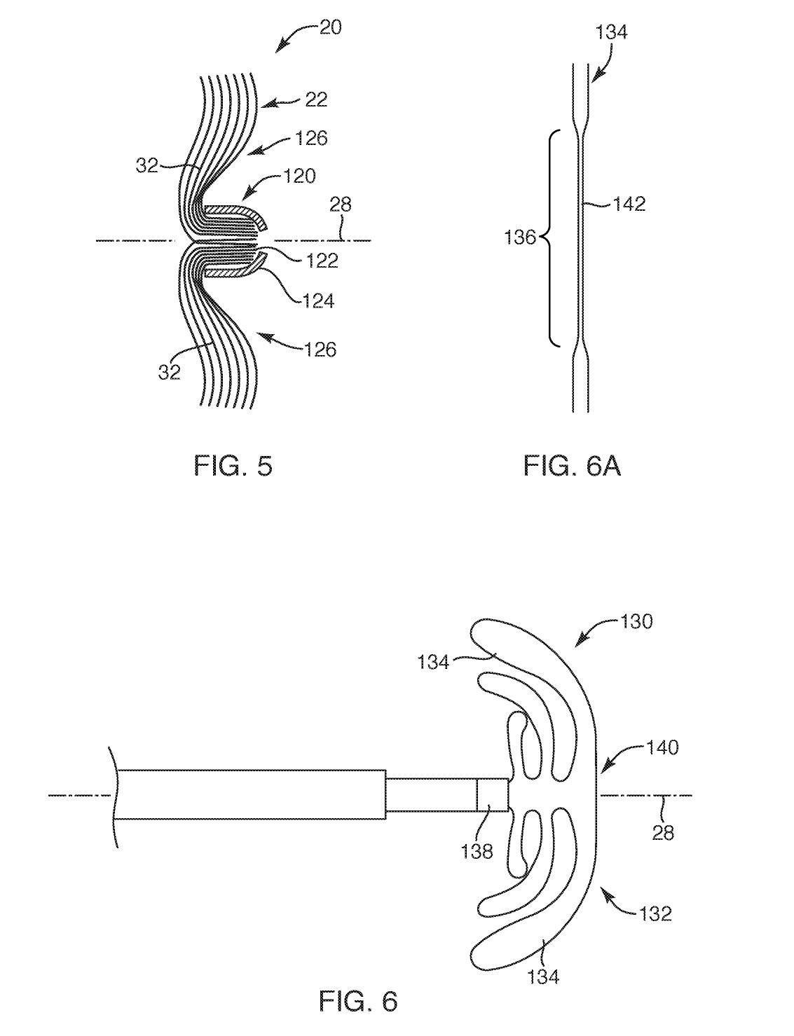

For example, with reference to FIGS. 1A and 2, the guidewire end portion 20 may be configured to expand to exhibit the one or more pad portions 30. FIG. 1A depicts the distal side surface 22 of one of the one or more pad portions 30 of the guidewire end portion 20. FIG. 2 depicts a simplified cross-sectional profile view of the one or more pad portions 30 (without some of the detail for simplicity purposes). For example, although not fully depicted in these views, the guidewire end portion 20 may be formed with and include multiple wires 32, as partially depicted in FIG. lA (see also FIGS. 4A, 4B, and 5), in a woven or braided configuration extending between a proximal end 34 and a distal end 36 of the guidewire end portion 20. Such multiple wires 32 may extend to proximate and form the one or more pad portions 30.

In one embodiment, the one or more pad portions 30 may extend to exhibit a disc-like structure. In another embodiment, the one or more pad configurations 30 may extend radially and may curve radially outward and proximally to exhibit a cup-like structure. The one or more pad portions 30 may include a first pad 38, a second pad 40, and a third pad 42. The first pad 38 may be a distal most pad and may be larger than the second and third pads 40, 42. The second pad 40 may be positioned between the first pad 38 and third pad 42. Further, the second pad 40 may be larger than the third pad 42. Each of the pads may be longitudinally aligned and spaced along the axis 28 so as to form an in-line pad configuration 50.

In one embodiment, the first pad 38 may extend radially outward so as to be radially longer relative to the axis 28 than the second pad 40. For example, the first pad 38 may extend radially outward and proximally relative to the axis 28 so as to surround the second and third pads 40, 42 with a first radial end portion 44 such that the first pad 38 extends in a cup-like configuration. Similarly, the second pad 40 may extend radially outward and proximally relative to the axis 28 to surround the third pad 42 with a second radial end portion 46 in a cup-like configuration such that the second pad 40 may nest within the first pad 38. The third pad 42 may extend radially outward and may extend distally at a third radial end portion 48 of the third pad 42 similar to a disc-like configuration such that the third pad 42 is surrounded by the second pad 40 and the first pad 38. In this manner, the in-line pad configuration 50 may exhibit longitudinally aligned pads along the axis 28 and may be positioned relative to each other in a nesting arrangement. Such nesting arrangement may include the proximal and distal sides of adjacent pads being in direct contact with each other or they may be slightly spaced, as depicted. If slightly spaced, upon a distal force being placed on the guidewire 12 and the guidewire end portion 20 being braced against tissue, the spacing may be minimized so that they may come in direct contact with each other.

Further, each of the one or more pad portions 30 may include a rear view profile (see FIG. 1A) with a circular or oval profile or periphery, or any other suitable shape, such as a hexagon, octagon, or decagon profile. Further, each of the one or more pad portions 30 may extend with a profile thickness 52 between rear and front sides (e.g., distal and proximal sides) of each pad. Such profile thickness may vary along a radial length of each pad. In one embodiment, as a force is applied to the guidewire 12 and upon the distal side surface 22 of the guidewire end portion 20 being positioned against tissue, the guidewire end portion 20 may be compressible such that a length between the proximal and distal ends 34, 36 of the guidewire end portion 20 may shorten. Further, the profile thickness 52 of each pad may further contribute to the compressible characteristic of the guidewire end portion 20. In other words, upon a distal force being placed on the guidewire 12, the compressible characteristics of the guidewire end portion 20 may effectively absorb and diffuse some of the distal force therein.

In another embodiment, upon the guidewire end portion 20 being deployed to the expanded position, the distal side surface 22 of the guidewire end portion 20 may include a lateral width 53 or diameter that is at least five times larger than a width 55 or diameter of the guidewire body 16. In another embodiment, the lateral width 53 of the distal side surface 22 may be at least six times larger than the width 55 of the guidewire body 16. In still another embodiment, the lateral width 53 of the distal side surface 22 may be at least eight times larger than the width 55 of the guidewire body 16 or at least ten times larger than the width 55 of the guidewire body 16. The width 55 or diameter of the guidewire body 16 may be about 0.035 inches, or within the range of 0.030 to 0.045 inches.

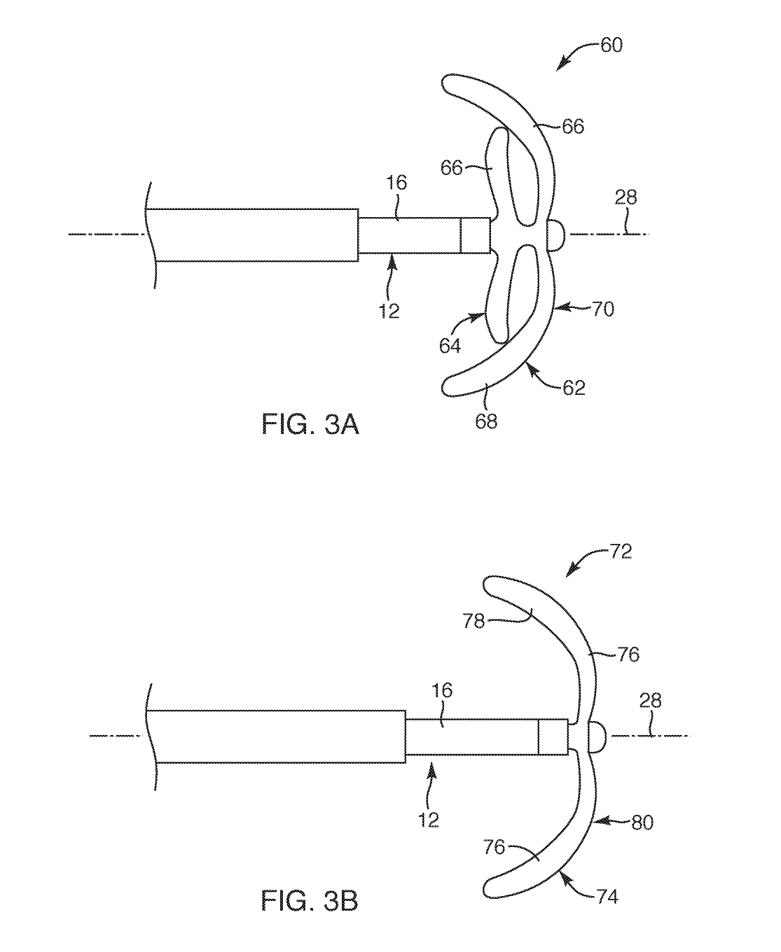

With respect to FIG. 3A, another embodiment of a guidewire end portion 60 coupled to the guidewire body 16 is provided. Similar to FIG. 2, FIG. 3A depicts a simplified cross-sectional view of a profile of the guidewire end portion 60. In this embodiment, the guidewire end portion 60 may include a first pad 62 and a second pad 64, the first pad 62 being more distal than the second pad 64. Further, the guidewire end portion 60 of this embodiment may be formed with multiple wires 66 to form and proximate the first and second pads 62, 64. The first pad 62 may be larger than the second pad 64. Further, the first pad 62 may extend radially relative to the axis 28 such that a first radial end portion 68 of the first pad 62 may extend proximally to surround the second pad 64. In this manner, the first pad 62 may include a cup-like configuration. In another embodiment, the first pad 62 may include a disc-like configuration. Further, the second pad 64 may include a disc-like configuration or a cup-like configuration that nests within the proximal side of the first pad 62. Further, similar to the previous embodiment, the first pad 62 includes a distal side surface 70 sized and configured to be positioned against tissue such that the guidewire end portion 60 of this embodiment may be braced against tissue without damaging the tissue and provide similar functionality as the previous embodiment.

With respect to FIG. 3B, another embodiment of a guidewire end portion 72 coupled to the guidewire body 16 is provided. In this embodiment, the guidewire end portion 72 may include a pad structure 74 in the form of a single pad. Similar to the previous embodiments, the guidewire end portion 72 may be formed to proximate and exhibit the pad structure 74 and may be formed with multiple wires 76 that may be woven or braided so as to proximate the pad structure 74. Such pad structure 74 may include a disc-like configuration or a cup-like configuration with a distal side surface 80 configured to be braced against tissue without damaging the tissue. Similar to previous embodiments, the pad structure 74 may include a radial end portion 78 that extends outward from the axis 28 and, in one embodiment, may extend radially outward and then proximally to exhibit the cup-like configuration. Further, the pad structure 74 of this embodiment may provide similar functionality as that described in previous embodiments for the guidewire end portion.

As set forth, the guidewire end portion, as described in the above embodiments and as depicted in FIGS. 2, 3A, and 3B, may be formed with multiple wires that may be woven or braided together such that the wires extend between first and second ends or proximal and distal ends. The termination of the multiple wires may be housed within, for example, proximal and distal hubs or tubular members.

With reference to FIG. 4A, a distal end portion 82 of the guidewire body 16 coupled to a proximal hub 84 of the guidewire end portion 20, such as the guidewire end portion depicted in FIG. 2. The guidewire body 16 may include a core portion 86 extending through a coil portion 88, the core portion 86 extending more distal than the coil portion 88. The core portion 86 may be elongated and extend a length of the guidewire body 16, the core portion 86 extending to a distal end. The core portion 86 may be a wire member, or the like, and may also be referenced as a core wire. The distal end of the core portion 86 or core wire may include a tip 90 having, for example, a sphere shape that is larger than a width or diameter of the core portion 86. Such tip 90 of the core portion 86 may be sized and configured to couple to the proximal hub 84 of the guidewire end portion 20.

The proximal hub 84 may include a tube 92 that defines a bore 94 therethrough. The tube 92 may house a portion of the core portion 86 and first ends 96 of the multiple wires 32 of the guidewire end portion 20. For example, the tube 92 may include a crimped portion 98 at a proximal portion 102 of the tube 92. The crimped portion 98 may be sized to surround the core portion 86 so as to constrict the tip 90 of the core portion 86 from withdrawing from the tube 92. In other words, the inner diameter of the tube 92 at a proximal end is less than the dimension of the tip 90 of the core portion 86 so that the core portion 86 may be coupled to the tube 92. At a distal portion 104 of the tube 92, the first ends 96 of the multiple wires 32 may be gathered together and positioned within the bore 94 of the distal portion 104 of the tube 92. In one embodiment, the first ends 96 of the multiple wires 32 may be coupled to the tube 92 with a second crimped portion 106 defined in the tube 92. In another embodiment, some or all of the first ends 96 of the wires 32 may be coupled to the tube 92 by, for example, through slots in the tube or welding, or any other suitable technique for coupling the first ends 96 or first end portions of the wires 32 to the distal portion 104 of the tube 92. With this arrangement, the guidewire end portion 20 may be coupled to a distal end portion 82 or distal end of the guidewire body 16.

With reference to FIG. 4B, another embodiment of the guidewire end portion 20 coupled to the guidewire body 16 is provided. As in the previous embodiment, the guidewire body 16 may include the core wire 86 surrounded by the coil portion 88, the core wire 86 including the tip 90 at a distal end thereof. Further, the guidewire end portion 20 may include a proximal hub 108, the proximal hub 108 including a proximal portion 110 and a distal portion 112 such that the proximal portion 110 may be coupled to the guidewire body 16 and the distal portion 112 of the proximal hub 108 may couple to the multiple wires 32 of the guidewire end portion 20. In this embodiment, the proximal hub 108 may include a filler coil 114 or tube positioned within the proximal portion 110 of the proximal hub 108 and around the core wire 86 such that the filler coil 114 is positioned proximal the tip 90 of the core wire 86. In one embodiment, such filler coil 114 may be configured to fill space in the proximal hub 108 so that the proximal hub includes an outside diameter similar to the outside diameter of the guidewire body 16. In another embodiment, the filler coil 114 may assist in preventing the tip 90 of the core wire 86 from decoupling from the proximal hub 110 of the guidewire end portion 20. Furthermore, the proximal hub 108 may include a first crimped portion 116 and a second crimped portion 118. The first crimped portion 116 may constrict the tip 90 of the core wire 86 from withdrawing from the proximal hub 108 and the second crimped portion 118 may couple the first ends 96 of the multiple wires 32 within the bore at the distal portion 112 of the proximal hub 108. In this manner, the guidewire end portion 20 may be coupled to the guidewire body 16.

With reference to FIG. 5, a distal hub 120 of the guidewire end portion 20, such as the guidewire end portion depicted in FIG. 2, is provided. The distal hub 120 may be sized and configured to couple to the second ends 122 of the multiple wires 32 of the guidewire end portion 20. The distal hub 120 may be a tubular member or the like that may include a crimped portion 124 to maintain the second ends 122 of the wires 32 in a gathered manner. In one embodiment, the whole tubular member of the distal hub 120 may be crimped for restraining the wires therein so that the length of the distal hub 120 may be minimized. In another embodiment, the second ends 122 may be coupled together by, for example, welding the second ends together and, further maintained together with the crimped tube of the distal hub.

Further, the distal hub 120 may be positioned relative to the distal side surface 22 of the guidewire end portion 20 so as to be recessed within the distal side surface 22. In other words, the multiple wires 32 extending to define the distal side surface 22 may define a recessed portion 126 that may be centrally located adjacent the axis 28 within the distal side surface 22 of the guidewire end portion 20. Such recessed portion 126 may be defined with second end portions of the multiple wires 32 that extend radially and proximally and then radially and distally to their distal second ends 122 to terminate within the distal hub 120. In this manner, the distal hub 120 may be positioned in a recessed manner relative to the distal side surface 22 of the guidewire end portion 20.

With respect to FIGS. 6 and 6A, another embodiment of a distal side surface 132 of a guidewire end portion 130 is depicted. In this embodiment, there is no distal hub, but rather, the multiple wires 134 extend across the distal side surface 132 along a middle portion 136 of the wires 134 such that the wires 134 overlap each other across or adjacent to the axis 28 of the guidewire end portion 130. Further, in this embodiment, the first and second ends of the multiple wires 134 extend within the proximal hub 138, instead of only the first ends of the multiple wires. Such first and second ends of the wires 134 may be gathered and held within the proximal hub 138, similar to that described in previous embodiments (see e.g., FIGS. 4A and 4B). As previously set forth, the middle portion 136 of the multiple wires 134 may extend over a central portion 140 of the distal side surface 132 and adjacent the axis 28 such that the multiple wires 134 may overlap along the central portion 140 of the distal side surface 132. To compensate for such overlapping of the wires 134 at the central portion 140, the middle portion 136 of the multiple wires 134 may be thinned to exhibit a thinned portion 142. FIG. 6A depicts the thinned portion 142 of one of the wires 134 at the middle portion 136 of the wire 134. Such thinned portion 142 may compensate for the overlapping of the middle portion 136 of the wires 134 at the central portion 140 of the distal side surface 132 to minimize bulging resulting from the overlapped wires 134. Further, such thinned portion 142 may also improve the ability of the wires 134 to bend and move between the constricted position and the expanded position without creating excessive stress in the wires 134. The thinned portion 142 at, for example, the middle portion 136 of the wires 134 may be thinned by employing a localized chemical process, such as chemical etching or electro-polishing, center less grinding, localized necking, or any other suitable process known in the art for thinning a portion of wire. With this arrangement, the thinned portion 142 minimizes potential bulging via the wires 134 overlapping at the central portion 140 of the distal side surface 132 of the guidewire end portion 130 and, further, allows the wires 134 to bend from the deployed or expanded position to the constricted or restrained position without creating excessive stress in the wires 134.

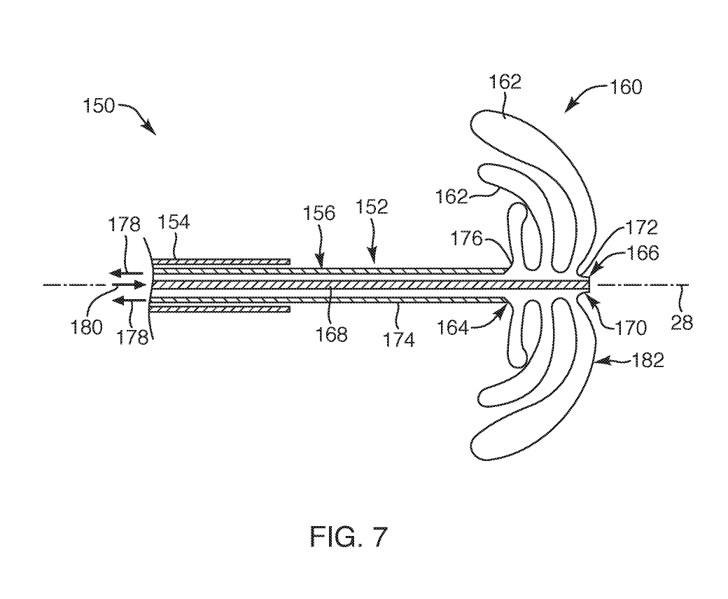

Referring now to FIG. 7, another embodiment of a guidewire system 150 with a guidewire end portion 160 is provided. Similar to previous embodiments, the guidewire system 150 may include a guidewire 152 and a catheter 154, the guidewire 152 having a guidewire body 156 with the guidewire end portion 160 coupled to a distal end portion or distal end of the guidewire body 156. In this embodiment, the guidewire end portion 160 may be actuatable such that the guidewire system 150 may be configured to actuate the guidewire end portion 160 between a constricted position (not shown) and an expanded position, the expanded position depicted in FIG. 7. As in previous embodiments, the guidewire end portion 160 may include multiple wires 162 extending with a woven or braided configuration between proximal and distal ends 164, 166 of the guidewire end portion 160. Further, the guidewire end portion 160 may be a super-elastic material so as to self-expand upon being deployed from the catheter 154 or sheath of the guidewire system 150. In one embodiment, the guidewire end portion 160 may be deployed in a controlled manner such that a physician may actuate components at a proximal end of the guidewire system 150 to actuate the guidewire end portion 160.

For example, in one embodiment, the guidewire body 156 may include a core member 168 or core wire with a distal end coupled to a distal hub 170 of the guidewire end portion 160. In one embodiment, the distal end of the core member 168 may be coupled to the distal hub 170 with second ends 172 of the wires 162 of the guidewire end portion 160 gathered together and also coupled to the distal hub 170. Further, the guidewire body 156 may include an elongated tube member 174 that may extend along the length (or portion of the length) of the guidewire body 156. The elongated tube member 174 may include a distal end that may be coupled to the first ends 176 of the wires 162 of the guidewire end portion 160. In one embodiment, the first ends 176 of the wires 162 may be coupled to a proximal hub, the proximal hub being interconnected to the distal end of the elongated tube member 174. In another embodiment, the distal end of the elongated tube member 174 may act as the proximal hub of the guidewire end portion 160.

The guidewire end portion 160 may be actuated by linearly moving the tube member 174 relative to the core member 168 along axis 28. In another embodiment, the guidewire end portion 160 may be actuated by linearly moving the core member 168 relative to the tube member 174. In either case, relative movement between the tube member 174 and core member 168 can actuate the guidewire end portion 160. For example, as indicated by arrow 178, the tube member 174 may be linearly moved proximally relative to the core member 168 to pull the wires 162 in the constricted position. Upon moving the guidewire end portion 160 to the constricted position, the catheter 154 may be moved distally over the guidewire end portion 160 to assist in maintaining the guidewire end portion 160 in the constricted position. Similarly, as indicated by arrow 180, the tube member 174 may be linearly moved distally relative to the core member 168 to move the wires 162 or guidewire end portion 160 to the expanded position. Prior to moving the guidewire end portion 160 to the expanded position, the catheter 154 may be moved proximally from over the distal end portion of the guidewire body 156. Once the catheter 154 is moved a sufficient distance proximally, the guidewire end portion 160 may still be in the constricted position and, upon actuation of the tube member 174 in a distal direction relative to the core member 168, the guidewire end portion 160 may be actuated to the expanded position so as to move to a pre-formed expanded state, as depicted in FIG. 7. Further, in another embodiment, the tension may be removed from the tube member 174 and the core member 168 such that, upon withdrawing the catheter 154 from the guidewire end portion 160, the guidewire end portion 160 may immediately self-expand to the expanded position as the catheter 154 is withdrawn from over the guidewire end portion 160. Further, in another embodiment, the actuatable guidewire end portion 160 may limit friction between the interior surface of the lumen of the catheter 154 and the guidewire end portion 160 as the guidewire end portion 160 is linearly moved through the lumen of the catheter 154. Such limiting of friction may be facilitated by maintaining proximal tension on the tube member 174 relative to the core member 168 as the guidewire end portion 160 advances or withdraws through the catheter 154. In this manner, the guidewire end portion 160 may be actuated by a physician. Further, the guidewire end portion 160 may include a distal side surface 182 for positioning and bracing against tissue such that the guidewire end portion 160 of this embodiment may provide similar functionality as set forth in previous embodiments.

The materials of the various embodiments set forth herein of the guidewire system may be formed of various medical grade biocompatible materials, as known in the art. For example, as indicated herein, the wires of the guidewire end portion may be formed of a super-elastic material. Such super-elastic material may include metallic or polymeric materials, such as Nitinol, or any other super-elastic alloy or polymer that may be suitable to enable the guidewire end portion to move between the constricted and expanded positions, set forth herein. The proximal and distal hubs of the guidewire end portion may be formed from stainless steel, or any other suitable metallic material. The guidewire body and catheter may be made of typical materials for these components, as known to one of ordinary skill in the art.

Further, as set forth herein, the wires of the guidewire end portion may be woven or braided together. Such woven or braided wires may be positioned with fixtures to the desired shape of the expanded position so that the positioned wires may then be heat-set in, for example, an oven or sand bath, thereby, setting the wires to self-expand to the desired expanded position or shape desired for the guidewire end portion, as known to one of ordinary skill in the art. Further, as known to one of ordinary skill in the art, other processes may be employed to the wires of the guidewire end portion, such as chemical etching and electro-polishing of the wires or other components of the guidewire end portion.

Now referring to FIGS. 8A through 8D, a method for employing the guidewire system with the guidewire end portion is provided. Although this method will be described relative to the embodiment of the guidewire system 10 and the guidewire end portion 20 of FIGS. 1, 1A, and 2, this method may be applicable for each embodiment described and depicted herein.

With respect to FIGS. 1 and 8A, in one embodiment, a physician may advance the catheter 14 or sheath of the guidewire system 10 through the vasculature of a patient, and through the aortic valve 183 to position a distal portion of the catheter 14 within the left ventricle 185 of the heart 187. Such advancing of the catheter 14 through the vasculature may be employed using a typical guidewire previously advanced to the left ventricle 185. The typical guidewire may then be withdrawn from the vasculature, leaving the catheter 14, as depicted in FIG. 8A.

With respect to FIGS. 1A, 2, 8A and 8B, the physician may then advance the guidewire 12 through the lumen of the catheter 14. The guidewire end portion 20 may be loaded within a loader member (not shown) to move the guidewire end portion 20 to the constricted position so that the guidewire end portion 20 may then be advanced through the proximal end 24 of the catheter 14 and moved distally toward a distal end 26 of the catheter 14. If the guidewire end portion is actuatable (as described in FIG. 7), the loader member may not be necessary to initially constrict the guidewire end portion prior to advancing through the catheter. Upon the guidewire end portion 20 being positioned adjacent the distal end portion of the catheter 14, the physician may position the distal end 26 of the catheter 14 adjacent an apex 189 of the left ventricle 185 or against tissue 191 adjacent the apex 189 of the left ventricle 185. At this point, the physician may move the catheter 14 proximally relative to the guidewire 12, or in the alternative, the guidewire 12 may be moved distally relative to the catheter 14. In either case, as the catheter 14 is moved or being withdrawn, the guidewire end portion 20 may become exposed and immediately self-expand to a radially enlarged or the expanded position (as portions of the guidewire end portion 20 are exposed from the catheter 14). The physician may then position the distal side surface 22 of the guidewire end portion 20 against the tissue 191 adjacent the apex 189 of the heart 187, as depicted in FIG. 8B.

At this juncture, the physician may withdraw the catheter 14 of the guidewire system 10 from the vasculature, as depicted by arrow 184 in FIG. 8C. The guidewire 12 and guidewire end portion 20 may maintain its position in the left ventricle 185 of the heart 187. Once the catheter 14 is removed from the vasculature, the physician may position a device catheter over a proximal end of the guidewire 12 and then advance the device catheter through the vasculature of the patient.

With respect to FIG. 8D, as set forth, a device catheter 186 may be advanced over the guidewire 12 until the distal end of the device catheter 186 is within the heart 187. At this stage, the device catheter 186 and implant 188 may be appropriately positioned, for example, relative to the aortic valve 183. In order to effectively position and deploy the implant 188 in the aortic valve 183, the physician may place a distal force, as indicated by arrow 190, on the guidewire 12. With the distal side surface 22 of the guidewire end portion 20 acting to spread, distribute and diffuse any force placed thereon upon being braced against the tissue 191 (thereby, limiting or preventing damage to the tissue), the physician can then focus on appropriately positioning the implant 188 in the aortic valve 183. In this manner, the physician can utilize the guidewire 12 as needed by placing the necessary distal force on the guidewire 12 to effectively position the implant 188 in the aortic valve 183 without concern of damaging the tissue 191 in the heart 187 due to the enlarged distal side surface 22 of the guidewire 12 and the shock absorbing or compressible, guidewire end portion 20. Once the implant 188 is released in the aortic valve 183, the device catheter 186 may be removed from the vasculature and the catheter 14 of the guidewire system 10 may then be advanced over the guidewire 12 and to the guidewire end portion 20 in the left ventricle 185. The guidewire end portion 20 may then be pulled into the catheter 14 into the constricted position, after which, the guidewire system 10 may be removed from the vasculature of the patient (see FIGS. 1 and 1A).

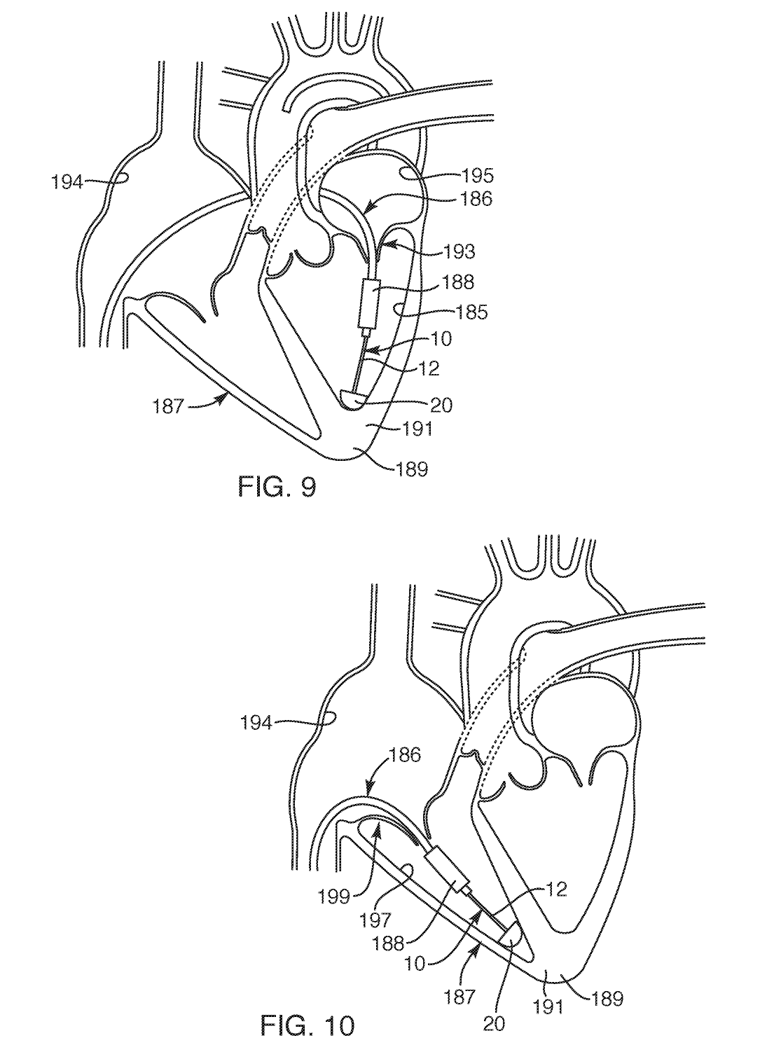

Similar to that described relative to FIGS. 8A through 8D, the guidewire system 10 may be employed with the device catheter 186 for positioning the implant 188 within a mitral valve 193 (see FIG. 9) and/or a tricuspid valve 199 (see FIG. 10). For example, with respect to FIG. 9, for purposes of employing the guidewire system 10 for implanting an implant 188 in the mitral valve 193, the guidewire system 10 may access the left ventricle 185 by advancing the guidewire system 10 through the septum of the heart 187 between the right and left atria 194, 195 and then advancing the guidewire system 10 through the mitral valve 193, as depicted, using typical interventional techniques known in the art. Upon positioning the guidewire 12 and guidewire end portion 20 in the left ventricle 185 through the mitral valve 193, the device catheter 186 may be advanced over the guidewire 12 so as to position the implant 188 adjacent the mitral valve 193. In this manner, the guidewire end portion 20 at the end of the guidewire 12 of the guidewire system 10 may be employed to be positioned and braced against tissue 191, such as the apex 189 of the heart 187, so that the implant 188 may be positioned and deployed, for example, in the mitral valve 193.

Further, for example, with respect to FIG. 10, in the case of positioning the guidewire system 10 in the right ventricle 197 for purposes of deploying an implant 188 in, for example, the tricuspid valve 199 of the heart 187, the guidewire system 10 may access the right ventricle 197 by advancing the guidewire system 10 into the right atrium 194 and through the tricuspid valve 199, using techniques known in the art. Upon positioning the guidewire system 10 in the right ventricle 197, the device catheter 186 may be advanced over the guidewire 12 to position the implant 188 adjacent the tricuspid valve 199. Similar to that described herein, the guidewire end portion 20 may then be employed for bracing against tissue 191 at the apex 189 of the heart to effectively facilitate deploying the implant 188 in, for example, the tricuspid valve 199.

Although the drawings describing the function of the guidewire system herein are focused on the left ventricle of the heart, the guidewire system set forth herein may be used for treatment in other anatomic locations where a less-traumatic guidewire placement is desired, such as the left atrium, right atrium, and right ventricle, and the arterial and venous vasculature. Further, the guidewire system of the present invention may be employed in non-cardiovascular locations, such as the digestive system, the urinary system, or other areas in which passage of a guidewire may be necessary or helpful.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention includes employing any portion of one embodiment with another embodiment, all modifications, equivalents, and alternatives falling within the spirit and scope of the invention as defined by the following appended claims.

What is claimed is:

1. A guidewire, comprising:
a guidewire body; and
a deployable guidewire end portion coupled to a distal end of the guidewire body, the deployable guidewire end portion having a self-expandable structure moveable between a constricted position and an expanded position.

2. The guidewire of claim 1, wherein the guidewire end portion comprises multiple wires, the multiple wires being woven together.

3. The guidewire of claim 1, wherein, upon the guidewire end portion moving to the expanded position, the guidewire end portion extends to proximate at least one pad structure.

4. The guidewire of claim 1, wherein, upon the guidewire end portion moving to the expanded position, the guidewire end portion extends to proximate multiple in-line pads.

5. The guidewire of claim 1, wherein the guidewire end portion comprises multiple wires extending to proximate at least one pad structure.

6. The guidewire of claim 1, wherein the guidewire end portion comprises a super-elastic material.

7. The guidewire of claim 1, wherein, upon the guidewire end portion moving to the expanded position, the guidewire end portion extends with a nesting pad structure.

8. A guidewire system, comprising:
a catheter; and
a guidewire having a guidewire body, the guidewire body extending between a proximal end and a distal end, the guidewire having a guidewire end portion, the guidewire end portion coupled to a distal end of the guidewire body, the guidewire end portion having a self-expandable structure moveable between a constricted position and an expanded position, the guidewire end portion moving to the constricted position upon being positioned within a lumen of the catheter, the guidewire end portion configured to self-expand to the expanded position upon being moved from within a catheter distal end of the catheter.

9. The guidewire system of claim 8, wherein the guidewire end portion comprises multiple wires, the multiple wires being woven together and extending to proximate a pad structure.

10. The guidewire system of claim 8, wherein, upon the guidewire end portion moving to the expanded position, the guidewire end portion extends to proximate multiple in-line pad structures.

11. The guidewire system of claim 8, wherein the guidewire end portion comprises a super-elastic material.

12. The guidewire system of claim 8, wherein, upon the guidewire end portion moving to the expanded position, the guidewire end portion extends to proximate a nesting pad structure.

13. The guidewire system of claim 8, wherein, upon the guidewire end portion moving to the expanded position, the guidewire end portion comprises a distal side surface having a lateral width that is at least five times larger than a width of the guidewire body.

14. A method of positioning a guidewire at a target location, the method comprising:
positioning a distal end of a catheter adjacent the target location;
advancing a guidewire through a lumen of the catheter, the guidewire including a guidewire body and a guidewire end portion, the guidewire end portion coupled to a distal end of the guidewire body; and
deploying the guidewire end portion from the distal end of the catheter such that the guidewire end portion includes a self-expandable structure to move from a constricted position within the catheter to a deployed, expanded position;
wherein, upon deploying the guidewire end portion, bracing a distal most side surface of the guidewire end portion against tissue adjacent the target location.

15. The method according to claim 14, wherein the deploying comprises deploying multiple wires of the guidewire end portion to extend and proximate a pad structure.

16. The method according to claim 14, wherein the deploying comprises deploying the guidewire end portion to expand and extend to proximate multiple in-line pad structures.

17. The method according to claim 14, wherein the deploying comprises actuating the guidewire end portion from the constricted position to the expanded position.

18. The method according to claim 14, wherein the deploying comprises deploying the guidewire end portion to expand and extend to proximate a nesting pad structure.

19. The method according to claim 14, wherein the deploying comprises deploying the guidewire end portion having the distal most side surface such that the distal most side surface includes a lateral width that is at least five times larger than a width of the guidewire body.

20. The method according to claim 14, wherein the bracing comprises placing a distal force on the guidewire to facilitate deploying an implant from a device catheter positioned over the guidewire.
